SEQUENCE LISTING (14 sequences)

SEQ ID NO: 1 - DNA, 20 bp, Human
gttggagctggtggcgtagg

SEQ ID NO: 2 - DNA, 20 bp, Artificial Sequence
  Nucleic acid probe
gttggagcttgtggcgtagg

SEQ ID NO: 3 - DNA, 20 bp, Artificial Sequence
  Nucleic acid probe
gttggagctagtggcgtagg

SEQ ID NO: 4 - DNA, 20 bp, Artificial Sequence
  Nucleic acid probe
gttggagctcgtggcgtagg

SEQ ID NO: 5 - DNA, 20 bp, Artificial Sequence
  Nucleic acid probe
gttggagctgttggcgtagg

SEQ ID NO: 6 - DNA, 20 bp, Artificial Sequence
  Nucleic acid probe
gttggagctgatggcgtagg

SEQ ID NO: 7 - DNA, 20 bp, Artificial Sequence
  Nucleic acid probe
gttggagctgctggcgtagg

SEQ ID NO: 8 - DNA, 20 bp, Human
gttggagctggtggcgtagg

SEQ ID NO: 9 - DNA, 20 bp, Artificial Sequence
  Nucleic acid probe
gttggagctggttgcgtagg

SEQ ID NO: 10 - DNA, 20 bp, Artificial Sequence
  Nucleic acid probe
gttggagctggtagcgtagg

SEQ ID NO: 11 - DNA, 20 bp, Artificial Sequence
  Nucleic acid probe
gttggagctggtcgcgtagg

SEQ ID NO: 12 - DNA, 20 bp, Artificial Sequence
  Nucleic acid probe
gttggagctggtgtcgtagg

SEQ ID NO: 13 - DNA, 20 bp, Artificial Sequence
  Nucleic acid probe
gttggagctggtgacgtagg

SEQ ID NO: 14 - DNA, 20 bp, Artificial Sequence
  Nucleic acid probe
gttggagctggtgccgtagg

(19) United States
(12) Patent Application Publication
(10) Pub. No.: US 2003/0219765 A1
(43) Pub. Date: Nov. 27, 2003

(54) Methods for evaluating cancer risk

(76) Inventor: Jose Costa, Guilford, CT (US)

Correspondence Address: WIGGIN & DANA LLP, ATTENTION: PATENT DOCKETING, ONE CENTURY TOWER, P.O. BOX 1832, NEW HAVEN, CT, 06508-1832, US

(21) Appl. No.: 10/271,179

(22) Filed: Oct. 15, 2002

Related U.S. Application Data
(63) Continuation-in-part of application No. 10/044,735, filed on Jan. 11, 2002.
Application No. 10/044,735 is a continuation of application No. 09/814,200, filed on Mar. 21, 2001.
(60) Provisional application No. 60/191,557, filed on Mar. 23, 2000.

(51) Int. Cl. C12Q 1/68; C12P 19/34

(52) U.S. Cl. 435/6; 435/91.2

(57) ABSTRACT

The present invention is directed to a method of evaluating the risk of cancer development in a patient, comprising the steps of: (1) providing from the patient a sample of material for which the risk of cancer development is to be evaluated; (2) quantitating the proportion of mutated alleles in the sample, relative to nonmutated alleles; (3) quantitating the degree of diversity of mutated alleles in the sample; (4) correlating the proportion of mutated alleles and the degree of diversity of mutated alleles; and (5) repeating steps (1) to (4) for a sufficient time to evaluate the risk of cancer development in the patient.

### CROSS-REFERENCE TO RELATED APPLICATIONS

This application is a Continuation-In-Part Application of U.S. Ser. No. 10/044,735 filed Jan. 11, 2002, which is a continuation of U.S. Ser. No. 09/814,200 filed Mar. 21, 2001, which claims the benefit of Provisional Application Serial No. 60/191,557, filed Mar. 23, 2000, all of which are incorporated by reference in their entireties.

### STATEMENT OF GOVERNMENT SUPPORT

This invention was made in part with government support under grant number CA-98-028 from the National Institutes of Health. The Federal Government has certain rights in this invention.

### BACKGROUND OF THE INVENTION

1. Field of the Invention

The present invention is directed to methods of evaluating cancer risk, and more particularly to methods of evaluating cancer risk by measuring the proportion of mutated alleles and the degree of diversity of mutated alleles in a sample from a patient.

2. Description of the Related Art

The factors that guide the evolution of a tumor share many similarities with macroevolution (Bodmer W. and Tomlinson I. Nature Medicine 5:11-2, 1999). During the earliest phases of the process, micro-clones of cells harboring mutations in genes implicated in the pathogenesis of tumors can be found to co-exist in tissues at risk for carcinoma (Moskaluk, CA, et al., Cancer Research, 57:2140-43, 1997; Deng, G, et al., Science 274:2057-59, 1996; Chaubert P, et al., Am. J. Pathology 144:767-75, 1994). Mutated alleles spread first within the clonal patches that constitute the developmentally regulated units of tissue architecture (FIG. 1). As shown in FIG. 1, in the colon, the physiologic deme is the crypt. Under normal circumstances, mutations accumulate randomly in each deme. When these mutations lead to favored growth of a single deme, yielding an oncodeme, the overall mutational complexity of the tissue is reduced. These changes may be impaired by morphologic criteria.

As indicated above, when a clone harbors a mutation in a gene implicated in the pathogenesis of cancer, it can be designated as an oncodeme. Increased risk of cancer has been correlated with certain diseases (precancerous conditions, e.g. atrophic gastritis) or to morphological alterations known as preneoplastic lesions (low, moderate and severe dysplasia). Extensive studies in epithelial organs have suggested that there is a dysplasia-to-carcinoma sequence representing the morphological manifestation of the emergence of a neoplasm. Yet, molecular genetic studies of coexisting early carcinoma and dysplastic lesions in tissues at risk for cancer suggest that diversity can be found among dysplastic lesions located in the vicinity of a tumor, and that a direct linkage between dysplasia and carcinoma is not easily demonstrated (Lin MC, et al., Am. J. Pathology 152:1313-8, 1998). Complete replacement of the precursor lesion by microinvasive carcinoma may in part explain this difficulty. However, a surprising finding of these studies is the demonstration of mutated cancer genes in lesions not known to carry an elevated risk of transformation, and even in morphologically normal tissues in the vicinity of a carcinoma. Thus, molecular preneoplasia does not have a necessary morphological correlate.

A diversity of mutations, both in terms of the genes affected and the mutated alleles, can be found in tissues known to be at high risk for carcinoma or already bearing a tumor. At least in two experimental rat models, N-methyl-nitrosourea (NMU) induced mammary carcinomas (Cha E.S., et al., Carcinogenesis 17:2519-24, 1996) and azoxymethane (AOM) related colonic carcinomas, mutations in the ras family of oncogenes occur in the absence of chemical mutagenesis. These results are of particular interest because at least some of the same mutated ras alleles can be found in the tumor, indicating they have been selected for during tumor formation.

Since it has been established that cancer results from genetic mutations and/or deletions, and that there exist normal mutations that are addressed by the cell itself (e.g., DNA repair or cell death), the challenge in developing methods for early cancer evaluation is to detect the emergence of significant mutations against a background of normal mutational complexity. Several patents have addressed this problem.

U.S. Pat. No. 6,428,964 discloses methods for detecting an alteration in a target nucleic acid in a biological sample. According to the invention, a series of nucleic acid probes complementary to a contiguous region of wild type target DNA are exposed to a sample suspected to contain the target. Probes are designed to hybridize to the target in a contiguous manner to form a duplex comprising the target and the contiguous probes “tiled” along the target. If a mutation or other alteration exists in the target, contiguous tiling will be interrupted, producing regions of single-stranded target in which no duplex exists. Identification of one or more single-stranded regions in the target is indicative of a mutation or other alteration in the target that prevented probe hybridization in that region.

U.S. Pat. No. 6,300,077 discloses methods for enumerating (i.e., counting) the number of molecules of one or more nucleic acid variant present in a sample. According to methods of the invention, a disease-associated variant at, for example, a single nucleotide polymorphic locus is determined by enumerating the number of a nucleic acid in a first sample and determining if there is a statistically-significant difference between that number and the number of the same nucleotide in a second sample. A statistically-significant difference between the number of a nucleic acid expected to be at a single-base locus in a healthy individual and the number determined to be in a sample obtained from a patient is clinically indicative.

U.S. Pat. No. 6,214,558 discloses methods for detecting in a tissue or body fluid sample, a statistically-significant variation in fetal chromosome number or composition to reliably detect a fetal chromosomal aberration in a chorionic villus sample, amniotic fluid sample, maternal blood sample, or other tissue or body fluid.

U.S. Pat. No. 6,203,993 discloses methods for comparing the number of one or more specific single-base polymorphic variants contained in a sample of pooled genomic DNA obtained from healthy members of an organism population and an enumerated number of one or more variants contained in a sample of pooled genomic DNA obtained from diseased members of the population to determine whether any difference between the two numbers is statistically significant. The presence of a statistically-significant difference between the reference number and the target number is indicative that the loci (or one or more of the variants) is a diagnostic marker for the disease. In a patient having a specific variant which is indicative of the presence of a disease-related gene, the severity of the disease can be assessed by determining the number of molecules of the variant present in a standardized DNA sample and applying a statistical relationship to the number. The statistical relationship is determined by correlating the number of a disease-associated polymorphic variant with the number of the variant expected to occur at a given severity level.

U.S. Pat. No. 6,143,529 discloses methods for detecting cancer or precancer by determining the amount of DNA greater than about 200 bp in length from a sick patient sample, and comparing the amount to the amount of DNA greater than about 200 bp in length expected to be present in a sample obtained from a healthy patient. A statistically significant larger amount of nucleic acids greater than about 200 bp in length in the patient sample is indicative of a positive screen.

All the above cancer detection methods are directed to detecting the presence or absence of mutated alleles, and developing a statistical correlation between the detected mutated alleles and the occurrence of cancer. However, strategies designed to simply detect the presence or absence of mutated alleles, even for genes of proven etiologic importance to cancer, most often fail to meaningfully discriminate patients with true premalignant lesions (i.e. ones that warrant therapy or increased surveillance) from patients with similar somatic changes who will never develop cancer. The reasons for this are manifold, relating primarily to the balance of host and environmental factors that modify the evolution of the clone that will become a given patient's cancer. Thus, there is a need in the art for early-detection strategies that will report not only the presence of genetic changes in a tissue or tissue surrogate, but will also detect, even against a constantly changing checkerboard of background mutations, if a true premalignant clone has emerged that is likely to progress. The present invention is believed to be an answer to that need.

### SUMMARY OF THE INVENTION

In one aspect, the present invention is directed to a method of evaluating the risk of cancer development in a patient, comprising the steps of: (1) providing from the patient a sample of material for which the risk of cancer development is to be evaluated; (2) quantitating the proportion of mutated alleles in the sample, relative to nonmutated alleles; (3) quantitating the degree of diversity of mutated alleles in the sample; (4) correlating the proportion of mutated alleles and the degree of diversity of mutated alleles; and (5) repeating the steps (1) to (4) for a sufficient time to evaluate the risk of cancer development in the patient.

These and other aspects will become evident upon reading the following detailed description of the invention.

### BRIEF DESCRIPTION OF THE DRAWINGS

The invention will be more fully understood from the following detailed description taken in conjunction with the accompanying figures in which:

FIG. 1 shows clonal expansion of individual deme into an oncodeme;

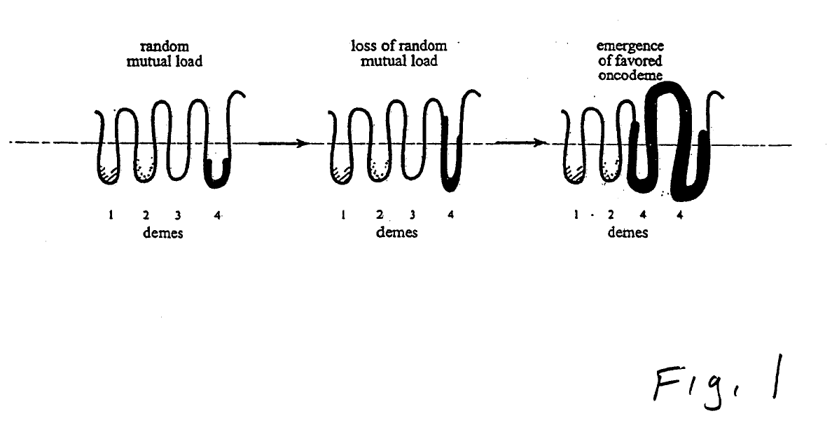

FIG. 2 shows the theoretical impact of clonal expansion of a deme on the mutational load distribution;

FIG. 3 shows how rolling circle amplification (RCA) functions as a generic reporter system for detection of immobilized analytes;

FIG. 4 shows a hybridization ligation system for detection of allele-specific reporter primers on DNA microarrays, based on RCA single molecule counting;

FIG. 5 shows in situ fiber-FISH hybridization in which allele-discriminating probes detect a point mutation at the G542X locus of the CFTR gene;

FIG. 6 shows an in situ hybridization experiment in which suitable allele-discriminating probes were used to detect a point mutation at the G542X locus of the CFTR gene;

FIG. 7 shows a schematic of a molecular beacon microarray comprising six different probe sequences;

FIG. 8 shows K-ras wild type and mutant alleles to be targeted for somatic mutation analysis in the ki-ras gene; and

FIG. 9 shows a composite chromosome map of WeGI 8341 vs. WeGI Female;

### DETAILED DESCRIPTION OF THE INVENTION

It has now been unexpectedly discovered that by monitoring the proportion of mutated alleles in a population of somatic cells, coupled with monitoring the degree of diversity at specific loci, it is possible to accurately evaluate the risk of cancer development in a patient. The method of the present invention thus measures (1) the proportion (e.g., number or frequency) of mutated alleles, and (2) the degree of diversity (e.g., distribution) of mutations at specific locations in the allele, and correlates this information over time to evaluate the risk of cancer in a patient. Thus, over time, using the method of the present invention, it is possible to screen cell samples for cancer and determine reliably and at an early stage whether a population of cells will likely develop cancer.

The method of the present invention is based on three discoveries: (i) all tissues harbor somatic mutations, with their prevalence dependent on the spontaneous mutation rate as modified by environmental exposure, DNA repair processes, and other factors; (ii) in the earliest stages of carcinogenesis, mutated alleles become dominant within a physiologic clonal patch (a deme). When mutations favor its expansion, the patch becomes an oncodeme; and (iii) the expansion of an oncodeme is the first cellular step in cancer evolution, and will be manifest even in a population of cells by a quantitative reduction in mutational diversity. Thus, by evaluating the quantity and distribution of mutations present in an ensemble of genes, it is now possible to evaluate the level of oncodeme expansion and thereby the risk of developing cancer.

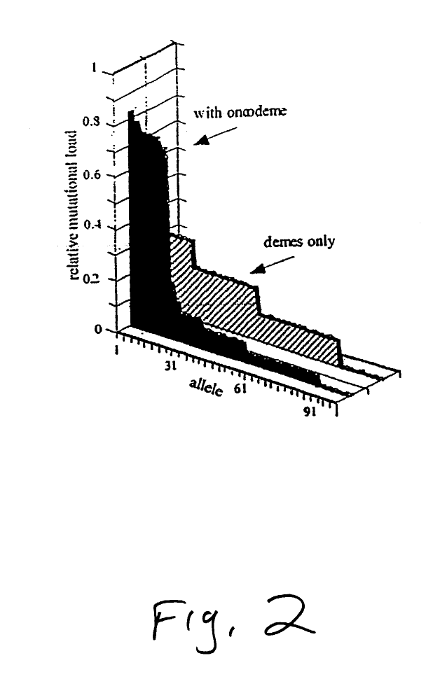

The method of the present invention utilizes a variety of highly sensitive methods of evaluating the quantity and distribution of mutations in a selected population of genes. By evaluating an adequate number of alleles over time, identification of emerging oncodemes is feasible. This can be illustrated by a simple theoretical example, in which a small population of somatic cells are evaluated for mutations at each of 100 alleles (FIG. 2). As shown in FIG. 2, 100 alleles were randomly mutagenized over a population of 10 demes, and the mutational frequency for the entire cell population plotted vs. allele. The expected distribution of mutations is broad for normal tissues (hatched); with the emergence of an oncodeme (solid), the distribution narrows significantly. The change in distribution is independent of any increase in mutational frequency in emerging clones, and in fact the two curves represented display no significant differences in the total mutational load.

The finding of somatic mutations is the result of random environmental mutagenesis followed by expansion of the allele within a physiological clone. The vast majority of clones will die before they accumulate additional mutations or before they expand further under the impulsation of selection. It is this fluctuation that is registered by the method of the present invention as random drift in the frequency of mutated alleles. Thus, for a randomly mutated normal population, the mutational load distribution is broad. Conversely, with the emergence of a single oncodeme that expands by 20 fold against the same background population, a loss of mutational load diversity becomes apparent. Therefore, by simultaneously mapping two or three altered cancer gene alleles to the geography of a tissue and allowing their concomitant expansion through time, it is possible to predict the location of where a tumor is likely to emerge. By repeatedly determining the proportion and diversity of mutated cancer gene alleles in fluids that sample a large population of cells from an organ in accordance with the method of the present invention, it is possible to evaluate the acquired cancer risk for the organ.

As defined herein, the term “allele” refers to any one of a series of two or more different genes that occupy the same position (locus) on a chromosome. The term “mutated allele” refers to an allele that possesses one or more nucleotide changes (point mutations) or a deletion or insertion of one or more nucleotides in its nucleic acid sequence. The phrase “proportion of mutated alleles” refers to the number of alleles that are mutated alleles, relative to the number of nonmutated (wild type) alleles.

The phrase “degree of diversity” refers to the type of mutational change displayed in a mutated allele. For example, a mutated allele may display three types of point mutations at a specific locus, relative to the wild type (wild type=T; point mutations are C, G, or A). A high degree of diversity would result from all three point mutations occurring at equal frequency (essentially randomly). A low degree of diversity would result if a specific point mutation becomes favored relative to the wild type.

To illustrate the degree of diversity, the following example is instructive. In the colon, where crypts are known to be clonal, exon 1 of the Ki-ras gene can be isolated as a PCR amplicon and analyzed by SSCP/sequencing. Microdissection of patches of 10 crypts by PCR/SSCP enables detection of mutated clones that have expanded to a minimal size of 600 cells or approximately one colonic crypt (in the rat intestine). Using this approach, normal, preneoplastic, and carcinomatous tissue, in normal and mutagenized rats may be studied. The prevalence of Ki-ras mutations found in the colonic epithelium does not differ significantly between non-mutagenized rats and mutagenized animals at 15 and 45 weeks after mutagenization, and that the same prevalence of Ki-ras mutations, about 4×10⁻³, is found in invasive AOM-induced tumors. However, whereas normal rats and rats early after mutagenesis show diversity of ras mutations, only one mutated allele is found in the tumor tissues and in normal tissues of rats 45 weeks after the administration of AOM. The allele selected for is consistent with the known effect of AOM (G to A transitions) and the short half life of this compound in the animal. The results observed in the group examined 15 weeks after mutagenesis are most simply explained if we posit that selection has contributed to the purification of a single allele in the tumors. (Table 1).

TABLE 1
Prevalence and distribution of Ki-Ras mutation in non tumoral tissues of
Fisher rat

| | % of Mutated Alleles | |
|---|---|---|
| Mutated Allele | Mutagenized* | Non-Mutagenized |
| GAT | 100 | 9.3 |
| TGT | 0 | 2.3 |
| GCT | 0 | 46.5 |
| GGT-GGT | 0 | 25.6 |
| Total Prevalence per thousand crypt | 2 | 5.2 |

*Five % of 311 tumors that did develop in the mutagenized rats harbored Ki-RAS mutation all in codon 12 with a G → A transition (GAT allele).

As defined herein, the term “correlating” refers to describing the relationship between the proportion of mutated alleles and the degree of diversity of mutated alleles for a selected allele. Such correlation may be displayed graphically, such as in FIG. 2 above, or may be displayed in tabular format. As defined herein, the phrase “sufficient time” refers to any time period required to assess the risk of cancer development with reasonable accuracy (generally on the scale of weeks to years).

As indicated above, the present invention is directed to method of evaluating the risk of cancer development in a patient comprising the steps of: (1) providing from the patient a sample of material for which the risk of cancer development is to be evaluated; (2) quantitating the proportion of mutated alleles in the sample, relative to nonmutated alleles; (3) quantitating the degree of diversity of mutated alleles in the sample; (4) correlating the proportion of mutated alleles and the degree of diversity of mutated alleles; and (5) repeating steps (1) to (4) for a sufficient time to evaluate the risk of cancer development in the patient. Each of the above steps is discussed in more detail below.

The monitoring of somatic mutation and genetic drift in human tissues requires a non-morbid method to sample the tissue at repeated intervals during the life of an individual. It is also desirable that the sample analyzed be as representative as possible of the entire organ or anatomical region that is being examined. Soluble DNA molecules present in biological fluids that drain or bathe the organs are excellent sources that meet the above criteria. By analyzing at the DNA rather than intact cells the sample thus better represents the entire cell population, rather than just cells physically close to the collection point.

In the method of the present invention, any body tissue or body fluid may be used as a sample source of DNA for organs or anatomical regions where mutations are to be quantitated. Examples of useful tissues or fluids include sputum, pancreatic fluid, bile, lymph, plasma, urine, cerebrospinal fluid, seminal fluid, saliva, breast nipple aspirate, pus, biopsy tissue, fetal cells, amniotic fluid, stool, and the like. Preferably, fluids derived from pancreas (ERCP aspirates), breast (nipple aspirates or nipple lavages), or colon (stool) are selected because of the possibility of obtaining surrogate fluids that contain cells and cellular material representative of the epithelial cell population from which cancer originates. Fluids can be collected from patients at high risk for cancers using protocols and methods well known in the art. For example, DNA can be isolated with relative ease from the fluid and cells obtained by endoscopic retrograde cannulation of the pancreatic duct. For breast, collecting nipple fluid should yield cells and biological material from a wide basin. Active aspiration of the nipple can consistently yield approximately 50 microliters of fluid from which cells, protein and soluble DNA can be obtained (Sauter E. R., Cancer Epidemiology, Biomarkers & Prevention 7:315-320, 1998), and which results in nanogram-range quantities of DNA. For colon, it is possible to perform cell brushings from small areas of mucosa during colonoscopy. Using this procedure, DNA samples from the interior of the colon may be obtained. DNA from colon may also be extracted directly from colon cells present in a stool sample.

All DNA extracted from the initial surrogate fluid samples can be quantitated and stored in aliquots containing diploid genome equivalents. Cytological specimens from brushings or fluids may be fixed in a fixative solution or on slides in a way that preserves them for the demonstration of point mutations. If tissues are to be used as a sample source, tissue samples may be obtained by laser capture microdissection. Following workup, each of the samples is then analyzed for point mutations and/or microdeletions using the methods described below. Although the method of the present invention is preferably implemented with DNA as a source for mutations, alternative nucleic acids, such as RNAs, may also be used in the method of the present invention. Accordingly, the invention is not intended to be limited by the source of nucleic acids in the samples.

In accordance with the method of the present invention, following sample isolation and preparation, the proportion of mutated alleles and the degree of diversity of mutated alleles in the sample are quantitated. In one embodiment, the step of quantitating the proportion of mutated alleles is done by first identifying the mutated alleles, relative to wild type (normal) alleles using techniques described below, and scoring (e.g., counting) the number of alleles with mutations. Similarly, in one embodiment, the step of quantitating the degree of diversity of mutated alleles in the sample may be performed by identifying the type of mutation relative to the wild type, and scoring that mutation. In general, the steps directed to quantitating the proportion of mutated alleles and the degree of diversity of mutated alleles in the sample may be performed by any method known in the art as long as it is a sensitive, quantitative, and efficient (i.e. high throughput) procedure that can simultaneously assess mutations in many alleles in cell populations the size of an oncodeme. Preferably, the selected method or methods will be capable of (1) detecting specific point mutations or microdeletions in a quantitative fashion; (2) testing a large number of samples; and (3) have a sensitivity at the level of detection of 1% of altered alleles in a background of wild type alleles. Examples of useful technologies for mutational analysis in accordance with the method of the invention include rolling circle amplification techniques, beacon array techniques, and comparative genomic hybridization. Each of these methods are described in more detail below.

In one embodiment, rolling circle amplification (RCA) techniques may be used to quantitate the proportion and degree of diversity of mutated alleles as described in Ladner et al., Laboratory Investigation 81:1079-1086 (August, 2001). Briefly, rolling circle amplification driven by a strand-displacing DNA polymerase can replicate circularized oligonucleotide probes with either linear or geometric kinetics under isothermal conditions (Lizardi, P. M. et al., Nature Genetics, 19:225-232, 1998). Using a single primer, RCA generates hundreds of tandemly linked copies of the circle in a few minutes. If matrix-associated, such as in arrays or cytological specimens, the DNA product remains bound at the site of synthesis where it may be fluorescently tagged, condensed and imaged as a point light source. Hybridization of a target sequence to immobilized and arrayed oligonucleotides can be visualized as single hybridization events and quantitated by direct molecular counting. When allele discriminating oligonucleotides are used to catalyze specific target-directed ligation events, wild type and mutant alleles can be discriminated as each allele generates a different fluorescent color signal when amplified by RCA. Thus, when used in an array format, RCA is particularly amenable for the analysis of rare somatic mutations and the study of mutational load.

In RCA, oligonucleotide probes are hybridized to complementary DNA targets and circularized by ligation. This ligation reaction may be exploited. for allele discrimination, or may be used to copy part of the target sequence into the circularized DNA. Using a single primer, complementary to the arbitrary portion of the circular DNA, a strand-displacing DNA polymerase (from phage Φ29) may be used to generate DNA molecules containing hundreds of tandemly linked copies of the covalently closed circle. In general, it takes less than 20 minutes to generate several hundred copies of the circular DNA template. When rolling circle DNA replication is carried out in the presence of two suitably chosen primers, one hybridizing to the (−) strand, the other to the (+) strand of the DNA, a geometrically expanding cascade of sequential DNA strand displacement reactions ensued, generating 10⁹ or more of copies of each circle in 90 minutes. This geometrically expanding cascade is called Hyperbranched Rolling Circle Amplification (HRCA). HRCA can be used to detect, among other things, point mutations at a specific locus of the CFTR gene in small amounts of human genomic DNA (Lizardi, P. M. et al., supra,). Like PCR, the Hyperbranched RCA reaction is capable of generating hundreds of millions of copies of a single DNA probe molecule. Therefore, HRCA is primarily useful for solution-based genetic analysis. For detection applications on the surface of microarrays, the linear, single primer reaction is a more attractive approach.

In one embodiment, RCA is useful for generation of individual “unimolecular” signals that may be localized at their site of synthesis on a solid surface. The DNA generated by a rolling circle amplification (RCA) reaction can be detected on a surface as an extended single strand, or as a condensed, tightly coiled “ball”. Cross linking reagents and fluorescence labeling may be used to permit observation of small spherical fluorescent objects of tightly condensed DNA arising from the amplification of a single circularized oligonucleotide (Lizardi, P. M. et al., supra). The individual signals are approximately 2 to 0.7 microns in diameter, and are easily imaged using an epifluorescence microscope with a tooled CCD camera.

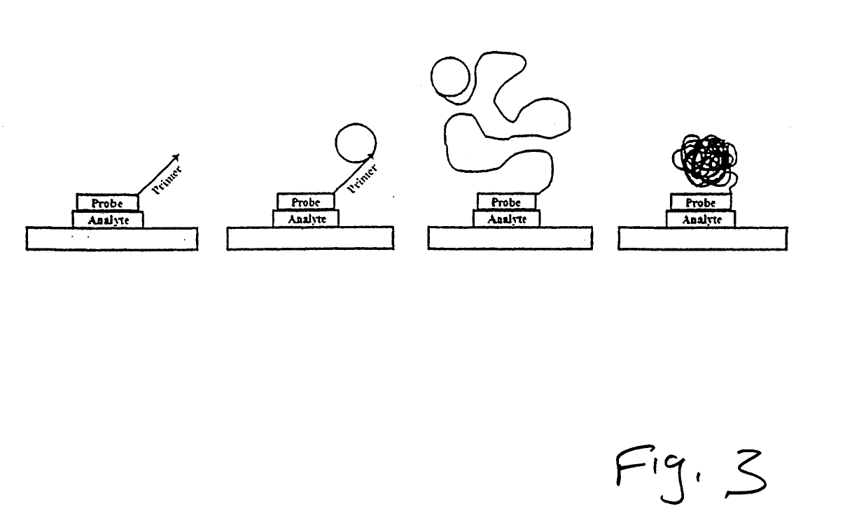

There are two alternative approaches for the use of localizable RCA signals in gene detection. The first approach consists of using a circularizable probe (called the Open Circle Probe) to interrogate the target sequence of interest (Lizardi, P. M. et al., supra). The second approach, shown in FIG. 3, consists of using a pre-existing circular DNA of arbitrary sequence, to extend a primer that is bound to a target on a surface of the primer is linked covalently to a detection probe, which defines target recognition specificity, while the circle is merely a reagent for a subsequent amplification reaction. As shown in FIG. 3, the probe-primer may contain any probe sequence. The circular DNA oligonucleotides, as well as the primers, contain arbitrary sequences. Because in this system the primer is a generic reporter that can be amplified by RCA, it is also possible to implement assays where the detection “probe” is an antibody capable of binding a specific antigen.

As mentioned above, RCA can be used for the generation of individual “unimolecular” signals that may be localized at their site of synthesis on a solid surface. Simple procedures known in the art using cross linking and fluorescence labeling permit observation of small spherical fluorescent objects that consist of a single molecule of amplified DNA. In this embodiment, multiple analytes may be detected using either DNA sample arrays, or oligonucleotide arrays. These types of applications require optimized surface chemistry, multicolor labeling protocols and DNA condensation methods, which are described below.

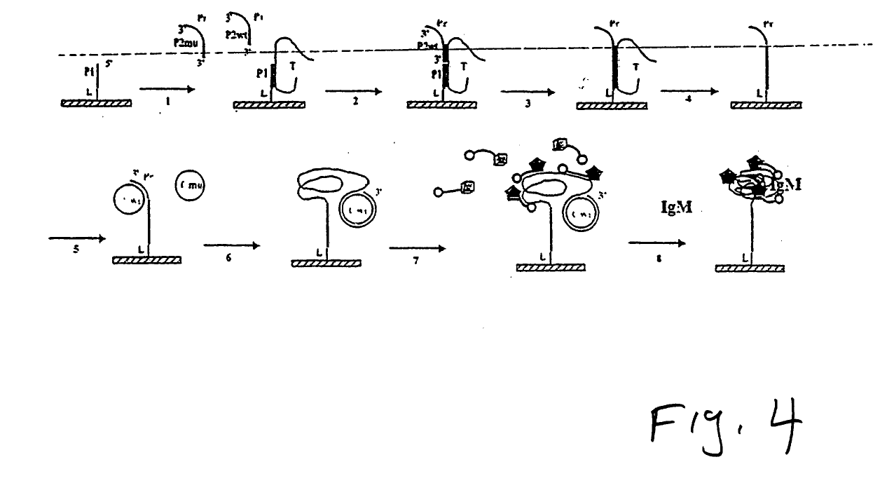

A strategy for detection of DNA targets using derivatized glass surfaces has been described and is known in the art (Lizardi, P. M. et al., supra). Briefly, the method exploits the capability for localizing RCA signals originating from single DNA primer molecules. This assay was used successfully to detect and quantify the frequency of a point mutation at the G542X locus of the CFTR gene by Single Molecule Counting. The assay measured the ratio of mutant to wild type strands at the G542X locus in genomic DNA samples of known genotype that had been constructed to simulate the presence of rare somatic mutations. Genomic DNA mixed in different ratios was amplified by PCR, and hybridized on slides with immobilized probes, in the presence of an equimolar mixture of two allele-specific probes in solution. After a hybridization/ligation step, ligated probe-primers were detected by RCA. The images showed many hundreds of fluorescent dots with a diameter of 0.2 to 0.6 microns, which were generated by single condensed DNA molecules. The ratio of fluorescein-labeled to Cy3-labeled dots corresponded remarkably closely to the known ratio of mutant to wild type strands, down to a value of 1/100. The Single Molecule Counting method is based on target-dependent ligation of reporter allele-specific probe-primers on a glass slide surface, and is shown in FIG. 4.

As shown in FIG. 4, a derivatized glass surface contains an oligonucleotide probe (P1) which is immobilized via a spacer (L); bound covalently on the glass. P1 is designed to form 22 to 39 base pairs with the DNA target, and the 5′ terminus of P1 contains a 5′-phosphate to permit ligation. This orientation is preferred because it eliminates the possibility of nonspecific priming by the 3′ end of P1, which could otherwise interact with the circular oligonucleotide templates used for RCA. In general, the method proceeds according to the following steps:

(1) A set of two allele-specific oligonucleotide probes (P2mu and P2wt) that are linked to different primer sequences (Pr, green or red) is allowed to hybridize with a DNA target (T);

(2) These probes, present in solution, hybridize to a 18 to 20 base sequence of the target adjacent to P1, with their 3′ end precisely in stacking contact with the 5′ end of P1, so that P1 and P2 may be ligated. P2-wt and P2-mu contain allele-specific bases at their 3′ ends. Both P2wt-Pr and P²mu-Pr contain at the opposite end a sequence that does not hybridize with the target, so that it may serve as a primer. These probe-primer molecules are synthesized a reversed backbone, and have two 3′ ends;

(3) After hybridization of the complementary allele-specific probe to target, which in the case shown is a wild type (green) sequence, a thermostable DNA ligase catalyzes the joining of P2wt-Pr to the immobilized P1 probe;

(4) The targets, excess probes, and any other molecules that are not covalently linked to the solid support are removed by very stringent washing;

(5) A mixture of two types of circular oligonucleotides, Cwt and Cmu are added, and they hybridize only to the complementary primer (Pr green). Thus, in the case illustrated only Cwt can hybridize;

(6) The covalently bound primer is extended by RCA, using the circular CTwt oligonucleotide as a template;

(7) The elongated DNA molecule is “decorated” by hybridization of DNP-oligonucleotide tags that harbor either fluorescein or Cy3 fluorescent labels. In the case shown, only the green tags are competent for binding, since the amplified circle only contains sequences complementary to the green tags;

(8) The amplified DNA product is condensed with anti-DNP IgM, forming a small globular DNA:IgM aggregate that contains green fluorescent tags.

The number of fluorescent objects of each color observed after imaging represents the number of DNA targets that participated in ligation reactions and generated covalently bound functional primers for RCA. The acronym for the process of condensation of amplified circles after hybridization of encoding tags is CACHET.

In situ methods may also be used to detect mutations in alleles. In one embodiment, DNA fibers may be used in conjunction with fluorescence in situ hybridization (FISH) techniques to detect mutations in alleles. Briefly, DNA fibers are prepared from cultured fibroblasts or lymphoblasts from normal individuals and individuals with homozygous or heterozygous mutations at the G542X locus of the cystic fibrosis gene using conventional DNA stretching techniques (Heiskanen M, et al., Genomics 30:31-36 (1995)). 1000-5000 cells in PBS buffer were spotted onto the end of a clean microscope slide, and the cells lysed for 5 minutes by the addition of an equal volume of 0.2% SDS. The slide was placed in a Coplin jar in a vertical position and the cell lysate allowed to dribble down the surface by gravity and then air dried. The sample was then fixed in methanol-acetic acid (3:1) for 10 minutes, washed, air dried and then treated with 0.1 mg/mi proteinase for 30 minutes, rewashed and air dried.

The design of the RCA probes used for allele discrimination at the G542X locus is as follows. The first oligonucleotide probe (P1) hybridizes to a 35-40 nucleotide sequence immediately upstream of the nucleotide to be integrated and acts as an “anchor” probe. The second oligonucleotide (P2) contains 16-20 nucleotides complementary to the target, a spacer region and a 20-28 nucleotide RCA primer sequence. The P2 probe contains two 3′-ends, by virtue of a change in backbone polarity within the spacer region of the molecule. One 3′-end of P2 is competent for ligation and contains an allele-discriminating nucleotide at the terminus while the other 3′-end is complementary to a preformed circular oligonucleotide to be amplified by RCA. Fiber-FISH is performed by hybridization/ligatide of a mixture of one P1 probe and two different P2 probes. Each P2 contains a terminal nucleotide complementary to the known alleles present at any given genetic locus and a different RCA primer sequence. After ligation, a mixture of two different circles are added, each circle being complementary to one of the RCA primer sequences on the P2 probes. Depending on the outcome of ligation, a different P2 RCA primer is immobilized and becomes competent for generation of a specific RCA signal. Wild type and mutant alleles are discriminated by the fluorescence color produced by the detector oligonucleotides subsequently hybridized to the RCA product. Addition of DNA polymerase serves two purposes: a) signal generation via RCA and b) stabilization of the probe duplex by extension of the 3′ end of the P1 anchor probe. By increasing the overall length of the P1:P2 ligation complex to 100 nucleotides or more by primer extension, fairly stringent washing conditions can be used post-amplification, with consequent reduction of background noise from non-specifically bound RCA primers.

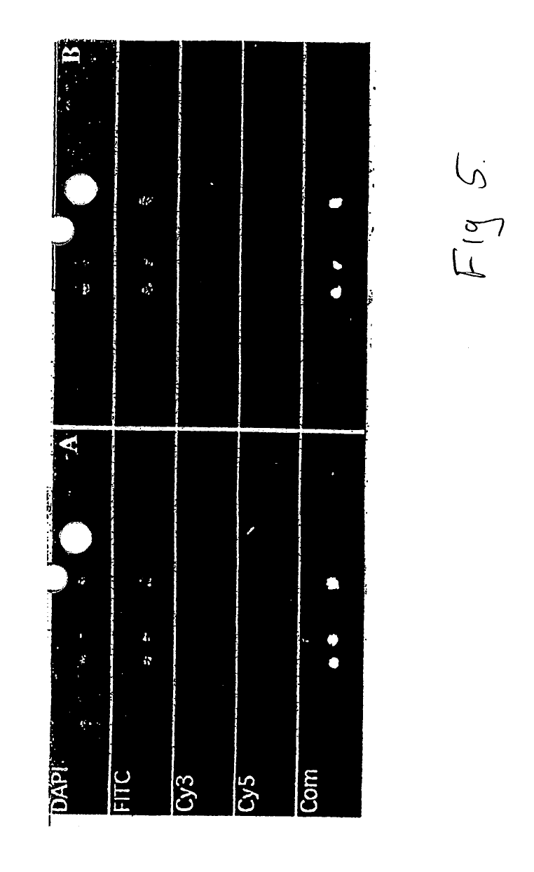

FIG. 5 illustrates the results typically obtained probing the G-542X locus. To better put this data in context, these allele discrimination experiments also included P1:P2 probe sets for the D508 and M1101K locus, which are both wild type in the individual examined. Briefly, two different lymphoblastoid cell lines were used, comprising homozygous wild type and homozygous mutant. (A) Images for the wild type cells; (B) mutant cells. All three RCA probes for the delta508, G542X, and M1101K loci were visualized with the fluorescein labeled decorator probe. The wild type delta508 allele is detected with Cy3, the G542X wild type is detected with CyS, and the M1101K is also Cy5. The mutant G542X allele is visualized by Cy3 labeling. The merged image (Com) shows that the wild type profile at all tree loci yields a yellow-white-white pattern, while the mutant profile shows yellow-yellow-white. The two top panels show the DAPI-stained DNA fibers. In FIG. 5, the RCA signals from these three loci can be visualized even in the DAPI image. Also, the D508 and G542 loci which are physically separated by 15 Kb are readily discriminated in these fibers. The physical distance between the G542X and the M1101K loci is 35 kb.

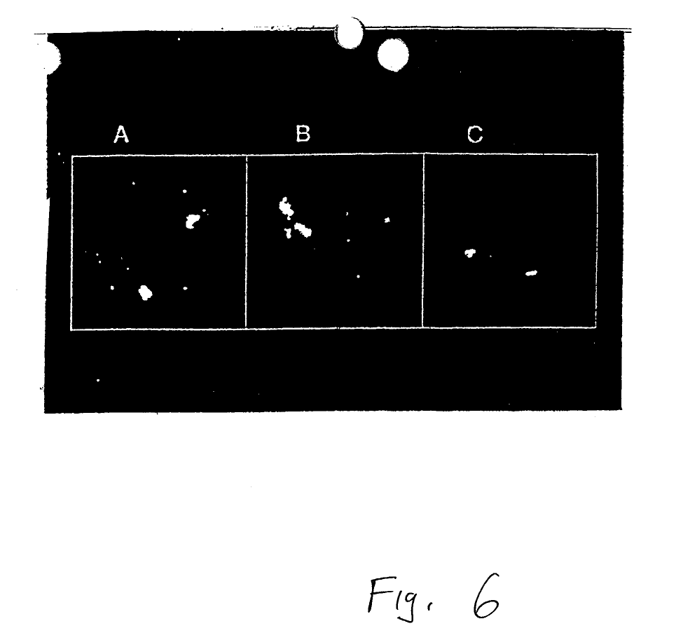

The same RCA probe design illustrated in FIG. 5 can be used to detect the different G542X alleles in interphase nuclei of cells derived from both normal individuals and cystic fibrosis patients. In one embodiment, the cells are hypotonically swollen, fixed in methanol-acetic acid (3:1), dropped onto microscope slides and hybridization/ligation/RCA reactions carried out as previously described. Typical results of raw, unprocessed images are illustrated in FIG. 6. Panel A shows two white signals in a wild type G542X cell; Panel B shows that 2 yellow signals are seen in a homozygous mutant cell. Panel C shows that cells from a G542X heterozygote exhibit one yellow and one white (mutant) signal while under the experimental conditions employed, RCA signals were seen in 70-80% of the cells examined. Most of the cells showed two signals per nucleus, however, a significant number of nuclei had 3 or 4 signals each. Cells with 4 signals had closely juxtaposed signal pairs (yellow-yellow or white-white; never yellow-white) suggesting that these cells were in G2 phase and the double signals were reflecting gene replication in S phase. The generation of gemini hybridization signals in interphase nuclei has been well documented previously and has been exploited to establish the replication timing of genes during progression through S phase (Selig, S., et al., EMBO J., 11:121701 (1992)).

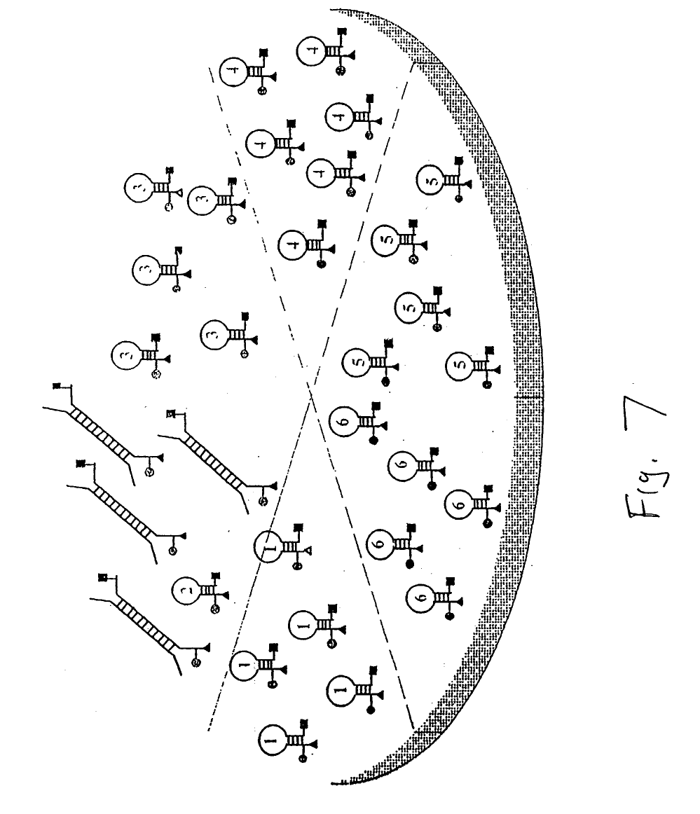

Molecular beacons are structured DNA probes that generate fluorescence only when hybridized to a perfectly complementary DNA target. The utility of these probes for the detection of specific sequences in PCR amplicons has been widely documented (Tyagi, S. et al., Nature Biotechnology 14:303-308 (1996); Tyagi, S., et al., Nature Biotechnology 16:49-53 (1998)). Molecular beacons may be immobilized on solid surfaces, where they function with the same excellent sequence specificity (Ortiz, E., et al., Molecular and Cellular Probes, 12:219-226 (1998)). Notably, immobilized beacons offer much larger potential for multiplexing relative to beacons used in solution. An important feature of molecular beacons is their improved capacity for allele discrimination, as compared to linear probes. The beacon stem provides an alternative stable structure that competes successfully with a mismatched hybrid, and thus the beacons remain in the quenched (closed) conformation even in the presence of target DNA capable of forming a mismatched hybrid. Allele discrimination ratios of 70:1 have been documented for many loci (Marras S. A. et al., Genet. Anal. 14:151-6 (1999); Bonnet, G. et al., Proc. Natl. Acad. Sci. USA (1999)). Molecular beacon arrays also offer advantages in terms of cost, reusability, and simplicity. A schematic of a hypothetical molecular beacon microarray is shown in FIG. 7. As shown in FIG. 7, probe sequence number 2 is shown interacting with a complementary DNA strand form a denatured PCR amplicon. Only beacon number 2 generates a fluorescence signal, while the other beacons remain in the closed conformation, and do not generate signals.

Immobilized molecular beacons are generally derived from oligonucleotides synthesized with a 3′-terminal DABCYL moiety, a reactive aminolinker side chain, a stem of 5 bases, a probe domain of 18 to 20 bases and a stem-complement of 5 bases, terminating with a fluorescent residue at the 5′-end. Some of the original molecular beacons utilized fluorescein as the fluorophore. However, dyes which are less susceptible to photobleaching are generally preferred. Most notable among these are the ALEXA dyes (Molecular Probes, Inc.) which combine high fluorescence yield with high resistance to photobleaching.

The oligonucleotide synthesis generally takes place in an automated synthesizer using standard phosphoramidite chemistry using standard reagents. Oligonucleotides are aliquoted on standard microtiter dishes at a concentration of about 200 μM. They are then dispensed as small droplets on the surface of activated glass slides (20 nanoliters per droplet) using the microarraying robot. Standard glass microscope slides are pre-activated with monomethoxysilane, generating a derivatized monolayer harboring the functional group 1,4-phenyler adiisothiocyanate. The primary amine in the second position of the molecular beacon oligonucleotide reacts with the derivatized glass surface, generating arrays with a high coupling efficiency (1×10¹¹ beacon molecules per square mm).

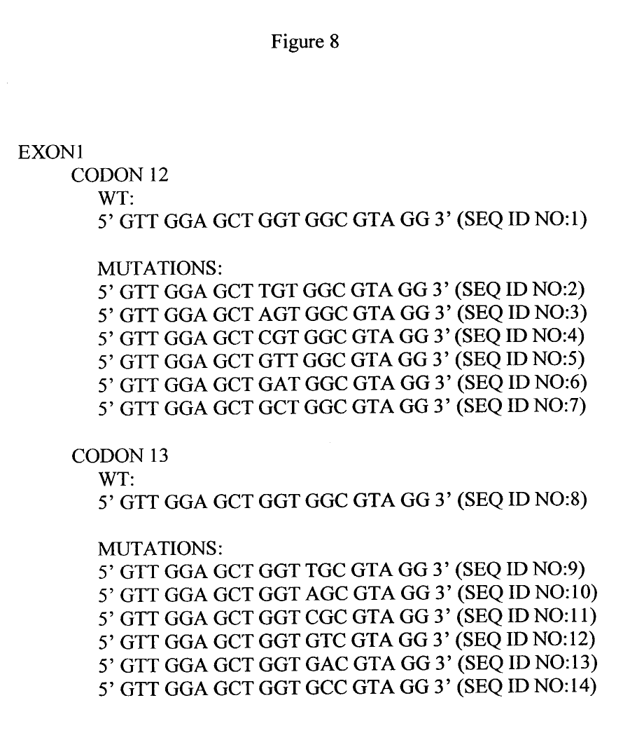

A total of 250 loci in the p53 gene will be targeted by 500 allele-specific, molecular beacon probes. The 250 loci will comprise those base positions where the highest frequency of mutation has been reported. For each locus, 250 wild type and 250 mutant-specific beacons are constructed and arrayed. To choose these loci, software and database tools available on the web (Cariello, N. F. et al., Nucleic Acids Res. 25:136-137 (1997); Béroud, C. et al., Nucleic Acids Res. 26:200-204 (1998); Hainaut, P., et al., Nucleic Acids Res., 26:205-13 (1998)) may be used. For the ki-ras gene, probes for the most commonly mutated loci can be constructed, corresponding to a total of 14 allele-specific probes (see FIG. 8). For N ras and H-ras, a total of 23 allele-specific molecular beacons can be constructed corresponding to the most commonly mutated alleles. An additional 234 allele-specific beacons can be constructed for other loci that are mutated frequently in cancer of the pancreas, breast, or colon. Finally, 13 beacons can be designed to probe known loci in lambda phage PCR amplicons that are added to the hybridization mixtures in order to serve as internal controls for monitoring the performance of the molecular beacon microarrays. The total number of beacons in a microarray is preferably 784 (=28*28).

A subset of the samples that have been genotyped using PCR and molecular beacon arrays will be further analyzed by in situ detection of point mutations using RCA-CACHET. This analysis will serve to a) confirm the genotype; b) in the case of samples where some tissue organization is preserved, obtain a precise localization of the mutant cells and indicate whether a clonal population of cells is apparent; c) in collaboration with other biomarker groups, ask whether or not the cells that display the mutant genotypes co-localize with any other novel (histological) marker for early neoplasia. Operationally, the in situ mutation analysis, as described above, requires prior knowledge of the mutant genotype to be probed for. The PCR-molecular beacon analysis will provide this information, and suitable probes will thus be synthesized. The RCA-CACHET method (Lizardi, P. M., et al., Nat. Gen. 19:225-232 (1998)) may be used with two different fluorescence labeling strategies. The simpler strategy involves single-color labeling of each probe (as defined by the sequence of the circular oligonucleotides used for RCA). This strategy may be employed for the simultaneous probing of as many as 6 different probes, using fluorescent dyes that are well resolved spectrally. A more complex strategy, with greater potential for multiplexing, involves the use of multicolor coding. Here each probe will be associated with a specific color combination, said combination resulting from the use of different combinations of arbitrary sequence tags in the circular oligonucleotides used for RCA. In some cases, it is desirable to work exclusively with the simpler, non-combinatorial scheme, since most FISH experiments will involve mutant genotypes that are already known, and most likely limited to a few mutations in any given sample. Nonetheless, it is worth noting that the combinatorial color coding scheme, when implemented with 5 color codes, will have the power for probing 31 mutant genotypes simultaneously.

Comparative genomic hybridization (CGH) has become a powerful tool for assessing chromosomal abnormalities (genetic losses and gains) in a broad spectrum of tumors. CGH has been used to determine genetic alterations in a variety of tumor types and at various stages of progression. However, the major limitation of CGH is the level of resolution obtained using metaphase chromosomes as the endpoint readout. Recently, it has been demonstrated (Pinkel, D., et al. Nature Genetics. 20:207-11 (1988)) that cohybridization of reference and sample DNAs to an array of cloned (and mapped) genomic DNA can provide higher resolution analysis of copy number variation in tumor specimens. In using such clone arrays and the inclusion of sufficient control parameters for hybridization efficiency and specificity, differences in fluorescent ratios of clones represented in the tumor DNA at one, two or three copies per cell could be detected.

The performance criteria for array CGH (A-CGH) are more stringent than those of related array-based methods for measuring levels of gene expression. Single copy gene changes relative to the normal diploid state must be detected as reliably as large copy number changes. Since the entire genome is used as a hybridization probe, it is between 10 to 20 fold more complex than those used to profile expressed sequences and it contains significant amounts of highly repetitive sequence elements. Pinkel, et al. (supra) added various amounts of 1 DNA to reference human genomic DNA to define the sensitivity and quantitative capability of their A-CGH protocol. Using cosmid, P1, BAC and other large insert clones as array targets, Pinkel, et al. demonstrated that the measured fluorescence ratios were quantitatively proportional to copy number over a dynamic range of 200-500 fold, beginning at less than 1 copy per cell equivalent.

In the method of the present invention, A-CGH is implemented according to the method of Pinkel et al., and using cosmid, P1 and BAC clones spanning the chromosomal bands, listed below, that undergo gains or losses with high frequency in the early stages of breast, colon or pancreatic carcinoma. Four specific chromosomal regions are particularly useful for this method: chromosome 3p (deleted in breast and colon), 17p (deleted in colon, pancreatic and breast) 18q (deleted in colon, pancreatic and breast) and 20q (amplified in breast, pancreatic and colon).

The hybridization of two different samples of genomic DNA (one tumor and one normal), each labeled with a different fluorophore, to an array of cDNA clones in order to establish their relative DNA copy number has recently been reported (Pollack, J. et al., Symposium on DNA Technologies in Human Disease Detection, San Diego, November 1998). These investigators were able to demonstrate an analytical sensitivity sufficient to detect a two-fold change in DNA copy number, equivalent to the detection of low level DNA amplification or allele loss. Significantly, this approach provides the opportunity to monitor gene expression and DNA copy number changes in the same sample. The method of the present invention implements a similar strategy using either cDNA clones or, preferably, synthetic oligonucleotides, to form an array of genes or ESTs from the chromosomal regions described above. The number of mapped cDNAs and EST markers has increased dramatically over the past few years thus making it feasible to synthesize defined oligonucleotide probes spanning large segments of the genome. A unique feature of the method of the present invention is the use of rolling circle amplification (RCA) technique in an immunodetection mode to markedly increase the sensitivity of hybrid detection. Genomic DNA from the tumor cells, e.g., a small set of cells constituting a potential oncodeme, can be labeled by nick translation or random priming with biotinylated nucleotides. Control reference cell DNA can be labeled similarly using digoxigenin nucleotides. Post-hybridization detection can be done using “immuno-RCA”, a method recently shown to be capable of visualizing single antigen-antibody complexes in a manner analogous to the detection of single DNA-oligonucleotide hybridization events. Antibiotin antibody can be covalently coupled to an oligonucleotide that will form the primer for RCA amplification of a preformed circle. Antibodies to digoxigenin can be labeled with a different oligonucleotide sequence that will prime RCA on a second circle sequence. The resultant RCA products, reflecting amplification from the hybridization of tumor DNA (biotin) or control (Digoxigenin) DNA, can be distinguished by using two RCA detector probes labeled with different fluors. Two color ratio imaging of RCA products should define the relative copy number of genes within the sample. Using immuno-RCA to visualize and count individual oligonucleotide-genomic DNA hybridization events should both enhance the sensitivity of detection of A-CGH and provide a higher resolution analysis than large clone arrays. As gene map densities increase, immuno-RCA should permit copy number ratio imaging on a gene by gene basis.

Oligonucleotide probes are generally selected by sequence analysis of chromosomal regions known to display loss of heterozygosity (LOH) or gene amplification in cancer lesions. Candidate sequences will be compared to Genbank entries using the BLAST program, in order to find sequence domains that represent unique, single copy sequences with no known homologues at other chromosomal loci. Only unique sequences will be selected for inclusion in the arrays. The length of the sequences will be 60 bases to permit very stringent washing after array hybridization.

The immobilization and arraying of hundreds of different probe molecules on solid supports is accomplished by covalent attachment of chemically synthesized oligonucleotides (Guo, Z. et al. Nucleic Acids Research, 22:5456 (1994)) in combination of robotics arraying. Microarrays are prepared by covalent binding of chemically synthesized oligonucleotides containing a primary amino group at the 3′ end, a spacer sequence of 15 thymidine residues, a-probe sequence (60 bases), and a free 5′-end. Oligonucleotides are aliquoted on standard microtiter dishes at a concentration of 200 μM. They are then dispensed as small droplets on the surface of activated glass slides (about 20 nanoliters per droplet) using the microarraying robot. The surface density of covalently bound probes can be determined by hybridizing a saturating amount of fluorescein-labeled oligonucleotides and measuring the fluorescence of bound DNA using a Fluorimager. The calculated densities range from 1×1010 to 1×1011 molecules per square mm. According to the method of the invention, the best results are achieved with a probe density of 5×1010 probes per square mm., which corresponds to a probe tile of approximately 45×45 Angstroms (area of approx. 2000 sq. Angstroms per probe).

It has been discovered that CGH signal enhancement by RCA enables the counting of single molecular hybridization events, and can yield precise fluorescence ratio determinations. In order to implement this enhancement, the following procedure is used. Human DNA is labeled by nick translation using either biotinylated (for normal tissue) or digoxygenin-derivatized (for tester tissue) deoxynucleotide triphosphates, and the hapten-labeled DNA is used for CGH on oligonucleotide microarrays. As mentioned above, to address the microarray hybridization sensitivity problem, a generic two-hapten scheme for the generation of enhanced fluorescent signals by RCA may be used. Signal enhancement is applicable to any experimental system that contains immobilized haptens, such as biotin and digoxygenin. The scheme is enabled by immuno-RCA, a novel paradigm for the detection of antibody molecules that enables single molecule detection. In immuno-RCA, antibodies for a specific antigen are coupled covalently to unique oligonucleotide primer sequence. Post antigen-antibody complex formation, the samples are incubated with circular oligonucleotides, washed, and then antibody detection is performed using RCA. Two model systems for immuno-RCA have been designed and tested, as shown in Table 2.

TABLE 2
Model systems for immuno-RCA

| Antigen | Immuno-RCA antibody |
|---|---|
| avidin | anti-avidin IgG |
| anti-dig IgG | anti-sheep-IgG |

As shown in Table 2, avidin is the first antigen, and the reporter system consists of a DNA primer coupled covalently to an anti-avidin antibody. This system has many potential applications, since it permits the indirect detection of biotin though an avidin bridge. The second antigen is a sheep anti-digoxygenin immunoglobulin, and the corresponding reporter system for detection consists of a DNA primer coupled covalently to an anti-sheep IgG. Biotin and digoxygenin can be immobilized on glass slides using covalent coupling. These haptens, present at high surface density, make the derivatized glass slide competent for strong binding of the two model antigens, avidin and anti-dig-IgG. Solutions containing known concentrations of the two antigens are spotted on the hapten-derivatized glass surface. Detection is performed in four steps: (a) binding of the antibody-DNA primer reporters followed by washing to remove unbound material; (b) binding of a mixture of two kinds of circular oligonucleotides (circ1, circ2) containing specific complementary sequences for primer binding; (c) addition of DNA polymerase to catalyze the RCA reaction, which generates tandemly repeated DNA copies of the sequences of circ1 and circ2; and (d) visualization of the amplified DNA by binding of two kinds of fluorescent oligonucleotide tags, one specific for the repeats of circ1, the other for circ2 repeats. The tags contain the haptenic group dinitrophenol (DNP), and one of two alternative fluorescent moieties (CY3, fluorescein). After binding of the specific tags, a multivalent anti-DNP IgM is added to cross link the long DNA molecules, effectively condensing the fluorescent tags into a single light source. Each molecule of antibody thus becomes associated with a fluorescent object that is visible under the light microscope as either a fluorescein or Cy3 signal.

Antibody-DNA conjugates may be prepared according to a published protocol with modifications to ensure high yield. The antibody may be cleaved into half molecules by mercaptoethylamine, while an aminated oligonucleotide is activated by the heterobifunctional reagent sulfo-SMCC. The half-antibody containing a free sulfhydryl is mixed with the activated oligonucleotide to form a covalent adduct joined by a thioester linkage. Solution assays performed in the presence of complementary circular oligonucleotides revealed that the adducts primed the synthesis of long molecules of single stranded DNA. This result demonstrates that antibody DNA adducts are competent for RCA in solution. Graded concentrations of avidin, diluted in human serum were spotted on the glass surface to explore the dynamic range immuno-RCA detection. When avidin was spotted at high concentration, the images obtained after immuno-RCA consisted of a large number of overlapping fluorescent objects. At even higher concentrations the fluorescence overlap was complete, and signals were strong enough for imaging and quantitation using a Molecular Dynamics fluorimager. By contrast, at lower concentrations of avidin the signals could be imaged in the light microscope as discrete fluorescent objects.

The two antigens, avidin and anti-dig IgG, were mixed in different ratios, diluted in human serum to simulate complex biological samples, and then spotted on glass slides. They were detected with anti-avidin-priml and anti-sheep-prim2. The immuno-RCA assay generated discrete fluorescent signals whose spectra consisted of either pure fluorescein or pure Cy3. The absence of signals with mixed spectra indicates that the dots are generated by single molecules of antibody bound to avidin or anti-dig IgG. In each case, the observed ratios of fluorescein dots to Cy3 dots correspond closely to the known input ratios of avidin to anti-dig IgO. Mixed signals are not observed, supporting the interpretation that each signal represents an individual antigen-antibody complex.

The demonstration of the detectability of single antigen-antibody complexes by immuno-RCA indicates that the application of this signal enhancement method to array CGH can provide a dramatic increase in sensitivity. By using immuno-RCA to generated two-color signals derived from biotin and digoxygenin labeling in the array CGH experiments, the need for whole genome amplification of tissue DNA is eliminated, with potential improvements in accuracy. Additionally, the use of Immuno-RCA signal enhancement permits the use of smaller tissue samples, which should increase the likelihood of detection of LOH.

As indicated above, in one embodiment, the step of quantitating the proportion of mutated alleles is done by first identifying the mutated alleles, relative to wild type (normal) alleles using techniques described below, and scoring (e.g., counting) the number of alleles with mutations. Similarly, the step of quantitating the degree of diversity of mutated alleles in the sample may be performed by identifying the type of mutation relative to the wild type, and scoring that mutation. Although simple scoring is described above, in some cases it may be desirable to apply statistical analysis to the data generated above. For example, an analysis of the data using log-linear models to describe the joint frequencies of mutations occurring at each site may be used to study the mutation patterns in selected samples over time.. Techniques to manipulate this data are known in the art (Zelterman, D. Journal of the American Statistical Association 82:624-629 (1987); Zelterman, D. Models for Discrete Data, chapter 6, Oxford University Press (1999)). Such an analysis may reveal the likelihood that mutations at certain loci are related to others. The subsequent outcome of developing cancer in those individuals screened may also be analyzed using survival analysis (time to diagnosis) and logistic regression (for any cancer diagnosis). The independent variables will include demographic variables such as age, smoking histories, family prevalence to cancer development, and the like. The genetic data can be summarized as the total number of mutations (mutational load) and as the specific loci that are mutated.

Following quantitation of the proportion of mutated alleles and the degree of diversity of mutated alleles, the data is correlated to determine the risk of cancer development. As indicated above, correlating means establishing a relationship between the proportion of mutated alleles and the degree of diversity of mutated alleles for a selected allele. In the method of the present invention, a preferred type of relationship is one in which, for a specific allele, there is an increase in the proportion of this particular allele, relative to the wild type, and a concomitant decrease in the diversity of mutations at that allele. In other words, a natural selection occurs such that a particular mutation becomes dominant and is preferred for a particular allele. Simultaneously, there may be a decrease in the mutational load of one or more other alleles, such that the total mutational load remains the same as a randomly mutated population (See FIG. 2).

The quantitating and correlating steps of the method of the present invention are repeated over a period of time and the particular locus is monitored for proportion of mutated alleles and degree of diversity. Preferably, the steps of the method of the present invention are repeated 2 to 10 times, and at intervals ranging from 6 times per year (every other month) once every two years, and more preferably twice per year to once per year. As indicated above, it is difficult to determine whether a particular mutated allele will mature into a malignancy by simply identifying the mutation because the background of normal mutational occurances and complexity significantly masks those true premalignant clones that are likely to progress into cancer. By repeating the steps of the method of the present invention over time, a pattern of identifiable alleles will emerge that are likely to progress into cancer. The data collected on each evaluation can be stored and compared over time to evaluate the risk of cancer.

It is worthwhile to note that even genes with no direct relevance to cancer are useful in this analysis, since to a first approximation somatic mutational events target all genes randomly. Thus while the method of the present invention focuses on genes of known tumor relevance, future applications of this method are likely to achieve ever increasing levels of sensitivity and discrimination by analyzing larger gene panels.

The methods of the present invention are useful for diagnosing and detecting early cancer development in any individual, and particularly those individuals who are predisposed to developing cancers, using noninvasive methods. By using the methods of the present invention, it is possible to monitor and follow the progression of cancer development in selected cells to observe what type of cancer develops so that an appropriate treatment can be implemented. The methods of the present invention are also useful for monitoring the progress and effectiveness of cancer therapies. For example, a patient on a chemotherapy could use the methods of the present invention to monitor how the chemotherapy treatment is affecting the mutated alleles that give rise to the cancer. In one embodiment, such a monitoring could show a gradual return from elevated proportions of mutated alleles and a low degree of diversity, to a background level of decreased proportions of mutated alleles and higher degree of diversity. The present invention is also useful for differentiating patients into risk groups (e.g., no risk, low risk, high risk, etc.), based on the outcomes of the methods of the present invention so that appropriate therapies can be prescribed.

### EXAMPLES

The following examples are intended to illustrate, but in no way limit the scope of the present invention. All parts and percentages are by weight and all temperatures are in degrees Celsius unless explicitly stated otherwise.

1. Sample Procurement

a) Pancreas

Pancreatic fine needle aspirations (FNAs) and common bile duct brushings are obtained from patients to be tested for cancer prevalence. Following the routine preparation of specimens for morphological analysis, the residual material, can be preserved and retained at 4° C. until further processing is desired.

b) Breast

Nipple fluid may be aspirated from patients undergoing stereotactic needle biopsy or needle localization biopsy for an abnormal mammogram. An average of 50 microliters of fluid can normally be obtained. These nipple aspirate fluids will be frozen and stored at −80° C. until processing.

c) Colon

Cellular brushings may be obtained from patients undergoing colonoscopy. Brush tips will be placed in ethanol and stored at 4° C. until further processing. Stool samples will be stored at 4° C. until lyophilization.

d) Preparation of cellular material from surrogate samples

For in situ assays, cellular pancreatic FNAs, common bile duct brushings, and colonic brushings in methanol or methanol-acetic acid are centrifuged at 1 85xg and fixed on glass slides by standard cytospin methods.

2. Laser Microdissection of Tissue and DNA Extraction for PCR Amplification

When surgically removed pancreatic, breast and colonic tissues become available from patients with matching surrogate samples, they are analyzed for mutational load and diversity using laser-capture microdissection. DNA from frozen, ethanol-fixed and formalin-fixed tissues may be routinely amplified using laser capture microscopy. Briefly, five-micron sections of tissue are cut and placed on glass slides, stained briefly with eosin and air dried. Sections are microdissected using a PixCell Laser Capture Microscope (LCM PXL-100, Arcturus Engineering, Inc., Mountain View, Calif.).

3. DNA Extraction

Cellular surrogate samples: Pancreatic FNAs, common bile duct brushings, and colonic brushings in ethanol or methanol are centrifuged at 185×g and DNA isolated from the pellets using the Easy DNA Kit for Genomic DNA Isolation (Invitrogen, Carlsbad, Calif.). Following ethanol precipitation, dried pellets are resuspended in TE buffer (10 mM TrisHCl, 1mM EDTA, pH 7.5) and quantitated by spectrophotometry (Genequant, Perkin Elmer, Inc.). Spectrophotometric quantitation is confirmed and DNA quality assessed by electrophoresis in 0.8% agarose and staining with ethidium bromide.

Stool: DNA from lyophilized and fresh samples is extracted using Catrimox-14 (Iowa Biotechnology Corp., Iowa, USA) according to manufacturer's protocol and resuspended in TE following ethanol precipitation.

Nipple aspirate fluids: DNA is extracted from nipple aspirate fluid using a sodium iodide-based DNA extraction kit (Wako Chemicals USA, Inc., Richmond Va.) following manufacturer's instructions and quantitated on 0.8% agarose gels by densitometry with comparison to placental DNA standards. Following quantitation, samples are stored at 4 degrees.

Laser-captured tissues: DNA is extracted from laser-dissected tissues by overnight incubation in Proteinase K or microwaving with GeneReleaser (BioVentures), 40 microliters final volume.

With the current protocols, it is possible to obtain between 0.1 and 15 micrograms of DNA. The sample is then assessed for the presence of mutated alleles by amplifying a 150 bp segment of exon 1 of the Ki-ras gene and the product is analyzed by SSCP. Three different concentrations are amplified independently and the bands compared and sequenced when necessary. This procedure permits the detection of 1% of a cell population harboring a clonally mutated Ki-ras allele. When the abnormally migrating band(s) represent 10% of the DNA migrating as wild type, the test is considered as indicating the presence of an expanded clone bearing an activating mutation in Ki-ras. In the presence of a mass detected by diagnostic imaging this is practically diagnostic of pancreatic cancer. It is important to emphasize that Ki-ras mutations have been detected in normal tissue and in dysplastic or preneoplastic pancreatic epithelium. In the case of analysis of the ERCP fluid as described above, the diagnostic value stems first from the fact that large amounts of DNA are analyzed, thus large number of cells (on average, the input for the PCR is 100 to 10,000 genome equivalents), and secondly from requiring a threshold of 10% clonally mutated alleles to consider a result as indicative of tumor. The molecular diagnostic assessment of ERCP fluids has proven a useful diagnostic adjunct to routine cytology (Table 3) (Dillon D A et al., Laboratory Investigation 77:37A (1998)). In addition, the mutations found in the fluid have been shown to correspond to the mutant alleles present in the tumors resected.

TABLE 3
Ki-ras mutational analysis in pancreatic FNAs and CBD brushings

| | Benign | Atypical | |
|---|---|---|---|
| | Morphology | Morphology | Malignant Morphology |
| Mutation | 2* | 7* | 8 |
| No mutation | 22 | 5 | 5 |
| Total | 24 | 12 | 13 |

*In clinical follow-up of these nine (9) patients, five (5) have a subsequent tissue diagnosis of adenocarcinoma, two (2) carry a clinical diagnosis of adenocarcinoma, one has died and one is still undergoing work-up.

4. Use of Molecular Beacon Microarrays.

DNA extracted from microdissected tissue may be amplified by polymerase chain reaction techniques (PCR) with the modification that one of the PCR primers will contain four phosphorothioate residues near the 5-end. After PCR, the amplicons are rendered single-stranded by digestion with T7 gene 6 exonuclease as described (69). A volume of 15 μl of solution containing the single-stranded PCR amplicons is then placed on top of a glass slide containing the molecular beacon microarray, covered with a plastic cover-slip, and hybridized at 55° C. for 30 minutes in a Hybaid Omnicycler slide incubation instrument. In addition to the tester PCR amplicons, a set of two additional PCR amplicons will be added as internal controls. These amplicons will be derived from the phage lambda genome, and will serve to monitor the performance of the molecular beacon array, which will include 10 probes for phage lambda. The incubation chamber is covered with aluminum foil to block room light. Fluorescence signals will then be imaged and quantified in a microarray reader.

5. Procedures and Protocols for RCA-Enhanced CGH

DNA is labeled by nick translation as described (Pinkel, D., et al. Nature Genetics 20:207-11 (1988)), except that the labels will consist of biotin-dUTP or digoxygenin-dUTP. Hybridization of the oligonucleotide arrays may be performed as described (Pinkel et al., supra). After washing, the slides are incubated with 5 μg/ml avidin and 10 mM sheep anti-digoxygenin IgG. After incubation for 20 minutes, the slides are washed with 2×SSC, 0.1% Tween-20 at 37° C. for 5 minutes and then air dried. Five μl of 15 nM rabbit anti-avidin IgG-pr1 conjugate mixed with 5 μl of 15 mM rabbit anti-sheep IgG antibody-Pr2 conjugate is applied to each microarray and incubated at 37° C. for 2 hours. The rabbit anti-sheep IgG antibody enables the detection of the sheep anti-dig antibody. The slides will be washed six times with 2×SSC, 0.1% Tween-20 and air dried. Five μl of 0.2 mM of the cir1 circular probe in DB1 buffer (2×SSC, 0.1% Tween-20, 3% BSA, 0.1% sonicated herring sperm DNA) is applied to each microarray. After hybridization at 37° C. for 20 minutes, the slides are washed with 2×SSC, 0.1% Tween-20 at 37° C. for 5 minutes and then air dried. RCA detection is performed as described (Lizardi, P. M., et al., Nat. Genetics 19:225-232 (1998)) with the following modifications:

Amplification with Sequenase: The reaction takes place in a volume of 40 μl in a buffer containing 40 mM TrisHCl (pH 7.5), 25 mM NaCl, 10 mM MgCl₂, 6.7 mM DTT, 3% v/v DM50, 200 μM dATP, dGTP, and dCTP, 100 μM dTTP, 10 μM biotin-dUTP. E. coli single-strand binding protein (SSB) is used at a concentration of 1.4 μM, and Sequenase 2.0 (Amersham Life Sciences) is at a concentration of 0.275 units/μl. Reactions are incubated at 37° C. for 15 minutes.

Fluorescence labeling: Oligonucleotide detector probes 18 bases long are hybridized to the RCA products, and each microarray is washed 1× with 2×SSC+0.05% Triton X-100 (SSC-T) at 45° C. for 2 minutes.

The labeled RCA products are condensed with 30 nM neutravidin at 37° C. for 20 minutes. Each slide is washed 2× with SSC-T, covered with antifade and imaged.

6. Methods for in Situ RCA-Cachet

RCA-CACHET may be performed using bipartite probes designed as described above. Methods for the generation of RCA signals in cytological preparations have been described (8). Currently these protocols permit the generation of signals in 70-80% of cell nuclei. We are currently refining these protocols in order to increase these levels to at least 85%-90% efficiency.

7. Reduction of Diversity

In the colon, where crypts are known to be clonal, exon 1 of the Ki-ras gene can be isolated as a PCR amplicon and analyzed by SSCP/sequencing. Microdissection of patches of 10 crypts by PCR/SSCP enables detection of mutated clones that have expanded to a minimal size of 600 cells or approximately one colonic crypt (in the rat intestine). Using this approach normal, preneoplastic and carcinomatous tissue, in normal and mutagenized rats have been studied. The results show that the prevalence of Ki-ras mutations found in the colonic epithelium does not differ significantly between non-mutagenized rats and mutagenized animals at 15 and 45 weeks after mutagenization, and that the same prevalence of Ki-ras mutations, about 4×10⁻³, is found in invasive AOM induced tumors. However, whereas normal rats and rats early after mutagenesis show diversity of ras mutations, only one mutated allele is found in the tumor tissues and in normal tissues of rats 45 weeks after the administration of AOM. The allele selected for is consistent with the known effect of AOM (G to A transitions) and the short half life of this compound in the animal.

Reduction of diversity of mutated alleles is shown above (Table 1) with respect to rats exposed to a mutagen that causes colonic tumors (AOM). The selection responsible for the emergence of a unique Ki-ras mutated allele in tumors was also found to be operating in non-tumoral oncodemes. The prevalence of mutations in the non-tumoral tissues of the mutagenized rats and the control rats was the same, five per thousand, but whereas the control rats harbored nine mutated alleles at codon 12 and 13, the mutatenized rats harbored a single mutation, GAT at codon 12. Thus, the random drift observed in the colon of controls was replaced by the emergence of a single GAT dominant allele in the non-tumoral regions of the colon.

It is possible to further demonstrate the reduction of diversity principle in the rat by repeatedly depleting the cell population of the large intestine and allowing it to regrow. This is accomplished by the iterative exposure of the animals to dextran sulfate, a chemical that kills intestinal cells but is devoid of mutagenic activity. It is observed that under the constant pressure to replenish lost cells, the random genetic drift at the Ki-ras locus is replaced by a single allele bearing a mutation in codon 13 (GGT-GGC). The fact that the emerging dominant allele differs from that seen under AOM mutagenesis is an indication that the natural allele is (GGT-GGC), whereas under AOM, a chemical carcinogen that specifically induces G to A transitions, it is the 12 GAT allele that emerges as dominant. Concomitantly with the restriction of Ki-ras alleles, the rats treated with dextran sulfate developed tumors. As the restriction at the Ki-tas locus was occurring the Ki-ras gene was wild type in the few tumors that appeared during the experiment. This result suggests that the method of the present invention can reveal a biological process that takes place in tissue and indicates the presence of a strong selection without being dependent on observing the gene or genes that will eventually be selected for in the tumor.

8. MLDA as a Biological Marker

The analyses described above show that a diversity of mutations in Ki-ras and p53 genes can be demonstrated in nipple fluid from two women not known at the time of analysis to harbor a breast cancer. However, no mutations are detected in soluble DNA obtained from human milk. Data based on a small sample of patients suggests a 10% prevalence for Ki-ras and a 5% prevalence for p53. In sharp contrast, control runs to correct for methodological errors (e.g., PCR-induced mutations) as well as the samples of milk revealed a prevalence below 1%. The high prevalence of mutations found in the soluble DNA recovered from fluids is perhaps due to the known pro-apoptotic effect of mutations in some genes, the ras family among others, or to massive DNA damage. Thus mutated DNA molecules may be over-represented in the DNA of fluids collecting debris issued from dying cells.

9. Whole Genomic Amplification and Array CGH

Isothermal amplification reactions based on strand displacement can be used to create replicas of entire genomes (Lage et al., 2002). For linear genomes, isothermal whole genome amplification (iWGA) proceeds via multiple initiation events driven by random primers, followed by DNA strand displacement and hyperbranching.

iWGA is catalyzed by Φ29 DNA polymerase, a highly processive enzyme with proof-reading activity. The error rate of Φ29 DNA polymerase has been reported to be in the range of 10⁻⁵ to 10⁻⁶ and the DNA amplified using this enzyme has been shown to be faithfully replicated. The yield of the iWGA reaction typically ranges from 200 to 10,000 fold, depending on the duration of the incubation. Typically, amplification reactions are incubated for 5 hours at a fixed temperature.

In array-CGH, experiments using DNA amplified by iWGA from as few as 500 cells of the breast cancer cell line BT474 (hybridized against amplified, normal human female DNA) we could demonstrate gains and losses of genes for almost all loci where changes had been detected in an identical experiment performed with unamplified DNA. Similar results were obtained with samples of 1000, and 500 cells from another breast cancer cell line MCF7. This type of array-CGH analysis may also be performed using DNA using DNA generated by iWGA from laser microdissected cells derived from a human breast cancer.

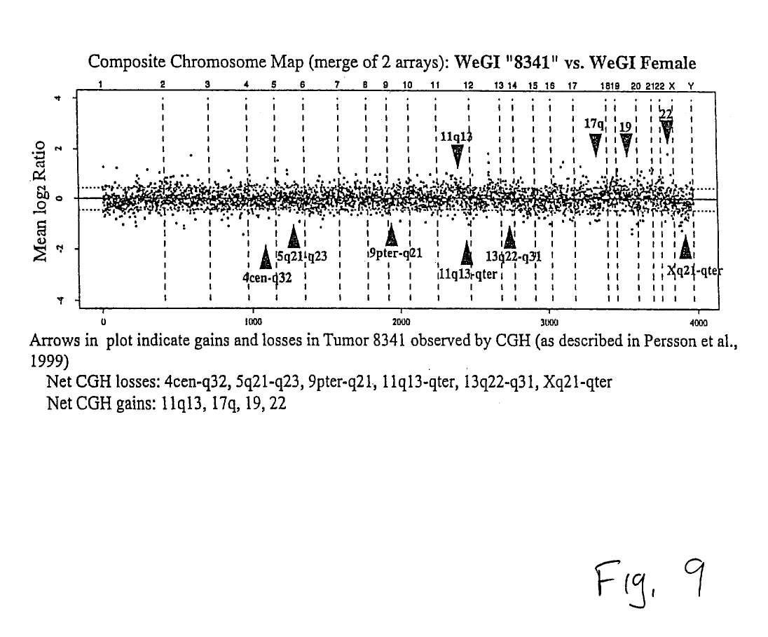

A frozen section of tumor sample 8341 was scraped with a needle. The contents of tumor cells in this section was around 95%. The DNA was extracted using MasterPure DNA purification kit, which ensures a DNA of high molecular weight. Approximately 25 ng of tumor DNA were amplified in a final volume of 100 μL using the conditions optimized for Whole Genome Isothermal Amplification with Bst polymerase. DNA from a female was also amplified following the same procedures with the purpose of being used as the reference DNA. After amplification, the samples were labeled with different dyes. Cy3 was used for the tumor sample, while Cy5 was used for the reference (female) DNA. Once labeled, both DNAs were mixed together with blocking Cot-1 DNA in hybridization solution, and dispensed over two identical arrays in the same slide. Hybridization was performed overnight. After hybridization, the slide was washed several times and scanned for both channels (dyes). The images were analyzed using Spot software, and the resulting data for both microarrays was merged into a single analysis. The results are shown in FIG. 9. As shown in FIG. 9, the analysis shoed that many alterations may be detected in regions previously described to be altered by CGH. Gains and losses are detected all over the genome, corresponding to genes over and under the confidence intervals.

10. In Situ Detection of Point Mutations using RNA

Messenger RNA is a more abundant target molecule than genomic DNA. Depending on transcriptional activity, specific mRNA sequences are represented in the cell as tens, hundreds, or even thousands of molecules. Based on published reports, kRAS mRNA may be present in the range of 50 to 150 copies per cell. Thus, detection of point mutations in situ using k-ras RNA as the molecular target can be a useful alternative to genomic DNA.

Incubation conditions have been described by Nilsson et al (Nucleic Acids Research 29:578-581, 2001). Using these conditions, k-ras exon 1 amplicons were generated by PCR from cell lines harboring known k-ras mutations (A549, LS180, SW480, and SW1116) using special primers with a T7 promotor sequence. The amplicons were then transcribed in vitro, using T7 RNA polymerase to generate RNAs of known allelic genotype. DNA probes specific for exon 1 were designed comprising two oligonucleotides that are ligated precisely at the site of each codon 12 point mutation. The in vitro generated mutant RNA transcripts were incubated in solution with pairs of DNA probes spanning the mutant sites (e.g., within the 3′-base of each of the probes paired at the exact position of the mutated allele.

In situ hybridization conditions for ligation mediated detection of point mutations in exon 1 of k-ras mRNA in cells and tissues was optimized. Paraformaldehyde fixation and mild protease treatment were found to yield optimal results. Control cells with normal k-ras genotype showed little background signal. However, specific RCA signals were observed when the mutant-specific probe was used for in situ hybridization in human tissue sections. A tumor harboring a codon 12 GGT to AGT mutation validated by PCR-SSCP analysis and DNA sequencing showed multiple signals.

While the invention has been described in combination with embodiments thereof, it is evident that many alternatives, modifications and variations will be apparent to those skilled in the art in light of the foregoing description. Accordingly, it is intended to embrace all such alternatives, modifications and variations as fall within the spirit and broad scope of the appended claims. All patent applications, patents, and other publications cited herein are incorporated by reference in their entireties.

What is claimed is:

1. A method of evaluating the risk of cancer development in a patient, comprising the steps of:
(1) providing from said patient a sample of material for which said risk of cancer development is to be evaluated;
(2) quantitating the proportion of mutated alleles in said sample, relative to nonmutated alleles;
(3) quantitating the degree of diversity of mutated alleles in said sample;
(4) correlating said proportion of mutated alleles and said degree of diversity of mutated alleles; and
(5) repeating said steps (1) to (4) for a sufficient time to evaluate the risk of cancer development in said patient.

2. The method of claim 1, wherein said sample is derived from pancreas cells or a fluid therefrom.

3. The method of claim 1, wherein said sample is derived from breast cells or a fluid therefrom.

4. The method of claim 1, wherein said sample is derived from colon cells or a stool sample.

5. The method of claim 1, wherein said quantitating step (2) and said quantitating step (3) are performed by rolling circle amplification.

6. The method of claim 1, wherein said quantitating step (2) and said quantitating step (3) are performed by comparative genomic hybridization.

7. The method of claim 1, wherein said quantitating step (2) and said quantitating step (3) are performed by molecular beacon assay.

8. The method of claim 1, wherein said quantitating step (2) and said quantitating step (3) are performed by single strand conformational polymorphism analysis.

9. The method of claim 1, wherein said quantitating step (2) and said quantitating step (3) are performed by laser capture microdissection.

10. The method of claim 1, wherein said quantitating step (2) and said quantitating step (3) are performed by hyperbranched rolling circle amplification.

11. The method of claim 1, wherein said quantitating step (2) and said quantitating step (3) are performed by fiber-based in situ hybridization.

12. The method of claim 1, wherein said quantitating step (2) and said quantitating step (3) have a sensitivity at the level of detection of 1% of said mutated alleles in a background of said nonmutated alleles.

13. The method of claim 1, wherein said correlating step comprises an increase in the proportion of a selected allele, relative to the wild type allele, and a decrease in the diversity of mutations of said allele.

14. The method of claim 1, wherein said repeating step is performed from 2 to 10 times.

15. The method of claim 1, wherein said method is repeated at intervals ranging from about 6 times per year to once every two years.

16. The method of claim 1 wherein said method is repeated at intervals ranging from about twice per year to about once per year.
